(19) United States
(12) Patent Application Publication
(10) Pub. No.: US 2025/0387434 A1
(43) Pub. Date: Dec. 25, 2025

(54) FORMULATION COMPRISING A HOMOGENEOUS POPULATION OF MESENCHYMAL STEM CELLS AND IMPLEMENTATIONS THEREOF

(71) Applicant: REGROW BIOSCIENCES PRIVATE LIMITED, Pune, Maharashtra (IN)

(72) Inventors: Satyen SANGHAVI, Pune (IN); Vinayak KEDAGE, Pune (IN)

(21) Appl. No.: 19/184,396

(22) Filed: Apr. 21, 2025

(30) Foreign Application Priority Data
Apr. 22, 2024 (IN) 202421031909

(51) Int. Cl. A61K 35/28; A61K 9/00; A61K 47/02; A61K 47/36; A61K 47/42; C12N 5/0775

(52) U.S. Cl. CPC A61K 35/28; A61K 9/0019; A61K 47/02; A61K 47/36; A61K 47/42; C12N 5/0665; C12N 5/0667

(57) ABSTRACT

The present disclosure relates to a formulation comprising: a) a population of mesenchymal stem cells (MSCs); and b) a carrier selected from serelaxin, Ringer's lactate solution, human serum albumin (HSA), dextran, heparin, hyaluronidase, or combinations thereof; wherein said population of MSCs is a homogeneous population having size in the range of 15-30 μm; wherein at least 50% of the MSCs express at least one marker selected from the group consisting of CD 90, CD73, and CD 105. The MSCs as part of the disclosed formulation exhibited 97% cell recovery and more than 90% cell viability.

### RELATED APPLICATION

The present application claims priority to Indian Patent Application number 202421031909, filed on Apr. 22, 2024, which is incorporated herein by reference in its entirety.

### SEQUENCE LISTING

This application contains a Sequence Listing which has been submitted electronically as an XML file. The Sequence Listing file is entitled PD054054US-CON_Sequence listing.xml, is 10 kilobytes in size, and has a creation date of Jul. 24, 2025. The information in the electronic format of the Sequence Listing is incorporated herein by reference in its entirety.

### FIELD OF INVENTION

The present disclosure relates to the field of mesenchymal stem cells (MSCs) and, particularly to, a formulation comprising MSCs and a carrier. The present disclosure further relates to a method of treating autoimmune or fibrotic disease using the formulation.

### BACKGROUND OF THE INVENTION

Dysfunction of the immune system plays significant role in the clinical manifestation of diseases, such as autoimmune diseases, inflammatory diseases like fibrotic diseases. Immunosuppressants have been widely used in the treatment of fibrotic diseases. Several varieties of drugs are available for treatment of autoimmune diseases like rheumatoid arthritis (RA), which includes immunosuppressants, steroid hormones, anti-rheumatic drugs, and anti-inflammatory drugs. But reported side effects of immunosuppressants and other drugs include immune deficiency, gastrointestinal tract disorders, hormonal disturbances, and complications in cardiovascular system; and sometimes patients become resistant to long-term treatments. Thus, the therapeutic approach for autoimmune disease, fibrotic disease and other such diseases demands an effective biological substitute, which is economical, long effective with no side effects.

Recently, Mesenchymal Stem Cells (MSCs) have been explored as an effective biological substitute in the treatment of several diseases. MSCs are adult stem cells that have the ability to self-renew and differentiate into multiple cell types. They are characterized by an extensive capacity for self-renewal, proliferation, potential to differentiate into multiple lineages and their immunomodulatory role on various cells. The majority of MSC products or therapies describe the use of cryopreservation conditions to store and transport the final product, which is usually thawed within a few hours prior to infusion. There are many challenges regarding the potential functionality of MSC products after preservation and thawing processes, particularly when bioactivity measurements are commonly conducted on MSCs before or without cryopreservation or following culture post-thaw.

The transport/storage conditions including temperature, chemical composition of the transport media and duration of transfer/storage are among the most critical factors that vary between cell production facilities and transplantation centres/laboratories. Transportation is challenging, requiring careful control of cell integrity, viability, and temperature to maintain the efficacy of the cellular product while conforming to important safety constraints.

Different types of carrier solutions have been tested previously, such as PBS, M199, culture medium, plasma lysate A, DMEM supplemented with 1% human serum albumin (HSA), NaCl etc., However, many of such carriers are not suitable for clinical and therapeutic treatment because they are not approved vehicles for safe infusion into patients.

### SUMMARY OF INVENTION

In an aspect of the present disclosure, there is provided a formulation comprising: a) a population of mesenchymal stem cells (MSCs); and a carrier selected from serelaxin, Ringer's lactate solution, human serum albumin (HSA), heparin, dextran, or combinations thereof; wherein said population of MSCs is a homogeneous population having size in the range of 15-30 μm; wherein at least 50% of the MSCs express at least one marker selected from the group consisting of CD 90, CD73, and CD 105.

In an aspect of the present disclosure, there is provided a method for preparing the formulation as disclosed herein, wherein the method comprises: mixing the plurality of MSCs with the carrier to obtain the formulation.

In an aspect of the present disclosure, there is provided a method of inducing tissue regeneration, comprising: administering to a tissue in need thereof the formulation as disclosed herein or obtained by the method as disclosed herein; wherein the tissue is selected from epithelial tissue; connective tissue like bone tissue, cartilage tissue, and elastic tissue; muscle tissue; or nervous tissue.

In an aspect of the present disclosure, there is provided a method of treating autoimmune or fibrotic disease in a subject, comprising: administering the formulation as disclosed herein or obtained by the method as disclosed herein to the subject.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1A depicts the flow cytometry results for UC-MSC characterization after storage/transport in Ringer lactate+1% HSA+Dextran 40 (1%), +Heparin (1000 U/ml)+Serelaxin (Ing/ml) as carrier at 0 hrs for CD73; CD90; CD105; CD34; CD45 and HLA-DR cell surface markers, FIG. 1B depicts the flow cytometry results for UC-MSC characterization after storage/transport in Ringer lactate+1% HSA+Dextran 40 (1%), +Heparin (1000 U/ml)+Serelaxin (1 ng/ml) as carrier at 6 hrs for CD73; CD90; CD105; CD34; CD45 and HLA-DR cell surface markers, FIG. 1C depicts the flow cytometry results for UC-MSC characterization after storage/transport in Ringer lactate+1% HSA+Dextran 40 (1%), +Heparin (1000 U/ml)+Serelaxin (1 ng/ml) as carrier at 12 hrs for CD73; CD90; CD105; CD34; CD45 and HLA-DR cell surface markers, FIG. 1D depicts the flow cytometry results for UC-MSC characterization after storage/transport in Ringer lactate+1% HSA+Dextran 40 (1%), +Heparin (1000 U/ml)+Serelaxin (1 ng/ml) as carrier at 24 hrs for CD73; CD90; CD105; CD34; CD45 and HLA-DR cell surface markers, and FIG. 1E depicts the flow cytometry results of Annexin V Analysis of UC-MSC after storage/transport in Ringer lactate+1% HSA+Dextran 40 (1%), +Heparin (1000 U/ml)+Serelaxin (1 ng/ml) as carrier for different time intervals (0 h, 6 h, 12 h and 24 h), in accordance with the embodiments herein.

FIG. 2A depicts the flow cytometry results for UC-MSC characterization after storage/transport in hyaluronidase as carrier at 0 hrs for CD34; CD45; HLA-DR; CD73; CD90; and CD105 cell surface markers, FIG. 2B depicts the flow cytometry results for UC-MSC characterization after storage/transport in hyaluronidase as carrier at 3 hrs for CD34; CD45; HLA-DR; CD73; CD90; CD105 cell surface markers, FIG. 2C depicts the flow cytometry results for UC-MSC characterization after storage/transport in hyaluronidase as carrier at 6 hrs for CD34; CD45; HLA-DR; CD73; CD90; and CD105 cell surface markers, FIG. 2D depicts the flow cytometry results for UC-MSC characterization after storage/transport in hyaluronidase as carrier at 12 hrs for CD34; CD45; HLA-DR; CD73; CD90; and CD105 cell surface markers, and FIG. 2E depicts the flow cytometry results of Annexin V Analysis of UC-MSC after storage/transport in hyaluronidase as carrier for different time intervals (0 h, 3 h, 6 h and 12 h), in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “osmolarity” as used herein refers to the number of particles of solute per liter of solution and is expressed as mOsm/L of solution.

The term “U/ml (units/ml)” as used herein refers to a unit of measurement that indicates the biological activity of substances like enzymes or hormones. The term “U/ml” may be used interchangeably with the term “IU/ml” (International units/ml) throughout the present disclosure.

Embodiments herein include a formulation comprising a population of MSCs and a carrier. MSCs are increasingly being used as off the shelf products for treating various diseases. The population of MSCs included in the present disclosure is a homogeneous population having size in the range of 15-30 μm and are therapeutically potent. Further, the MSCs secrete biologically active substances that have the paracrine ability called secretome, which is composed of cytokines, chemokines, growth factors, proteins, and extracellular vesicles. Specifically, the secretome of the MSCs population comprises VEGF and IL-10 that produce different effects leading to the process of therapeutic action of MSCs-derived secretome when transplanted to a patient.

Despite the increased demand for therapeutic MSCs, there is also a continued challenge of formulating the MSCs in appropriate carrier solutions for clinical and therapeutic treatment, ensuring safe infusion into patients. Also, choosing appropriate carriers is essential for the control of cell integrity, viability, and temperature to maintain the efficacy of the cellular product while conforming to important safety constraints. Based on the theory of cell ions and osmotic homeostasis, cell viability is strongly regulated by the osmolality and electrolyte concentration of the extracellular environment. Accordingly, embodiments herein provide a formulation comprising a population of MSCs and a carrier selected from serelaxin, Ringer's lactate solution, human serum albumin (HSA), heparin, dextran, hyaluronidase, or combinations thereof for therapeutic applications.

The Ringer's lactate solution is a mixture of not only sodium and chloride ions but also potassium ions and calcium ions, mimicking the extracellular fluid in the human body. The Ringer's lactate solution maintains high cell viability, cell attachment and recovery after 24 h of storage. Heparin aids in decreasing the formation of blood clots, while delivering the MSCs and thereby improves the effectiveness of cell therapy. Dextran 40 ensures slow release of the MSCs, thereby enabling a more gradual delivery of the MSCs at the target site and potentially improving the intended therapeutic effect. Hyaluronidase helps in the dispersion of the therapeutic agent, along with ensuring patient safety and comfort. Thus, the specific combination of serelaxin, Ringer's lactate solution, human serum albumin (HSA), heparin, and dextran ensures improved cell viability and recovery even after storage and thaw cycle. Embodiments herein also provide a method of preparing the formulation. Further, embodiments herein also include a method of inducing tissue degeneration and a method of treating autoimmune or fibrotic disease in a subject using the formulation.

Embodiments herein provide a formulation comprising a population of MSCs and a carrier. The term “mesenchymal stem cell” or “MSCs”, as used herein, refers to cell population of multipotent cells. According to the International Society for Cellular Therapy (ISCT), the criteria to define MSCs are their plastic-adherent ability; the high expression of positive markers, such as CD105, CD73 and CD90, as well as the lack of expression of negative markers, such as CD45, CD34, CD14/CD11b, CD79a/CD19 and HLA class II. The MSCs, according to embodiments herein, are characterized by the expression of one or more cell surface markers selected from CD73, CD90, or CD105. In further embodiments herein, the MSCs may be characterized by lack of expression of one or more of the markers selected from HLADR, CD34, or CD45.

In an embodiment, at least 50% of the MSCs express at least one marker selected from the group consisting of CD 90, CD73, and CD 105. In another embodiment, at least 50%, at least 55%, at least 60%, at least 65%, at least 70%, at least 75%, at least 80%, at least 85%, at least 90% or at least 95% of the MSCs express at least one marker selected from the group consisting of CD 90, CD73, and CD 105.

In an embodiment, less than 1% of the MSCs may be characterized by lack of expression of one or more of the markers selected from HLADR, CD34, or CD45. In another embodiment, less than 1%, less than 0.9%, less than 0.8%, less than 0.7%, less than 0.6%, less than 0.5%, less than 0.4%, less than 0.3%, less than 0.2%, or less than 0.1%, of the MSCs may be characterized by lack of expression of one or more of the markers selected from HLADR, CD34, or CD45.

The MSCs may be obtained from commercial sources or, alternatively, derived from tissues of mammals.

In an embodiment, the MSCs are derived from umbilical cord tissue, cord blood, adipose tissue, bone marrow, or dental pulp, preferably derived from Umbilical Cord Tissue (UCT) of mammals, in particular humans. HUC-MSCs are one of the most widely used MSC populations due to their therapeutic/regenerative properties and have advantages compared with MSCs from other sources.

The MSCs isolated from UCT may be a heterogeneous population of MSCs. The term “heterogeneous population”, as used herein, refers to a mixed population of small, medium, or large sized MSCs. According to embodiments herein, the MSCs having medium size may be expanded and produced using the methods generally known in the art.

In an embodiment, the homogeneous population of MSCs is obtained by artificial intelligence driven microfluidic sorting of culture expanded MSCs. In another embodiment, the homogeneous population of MSCs is obtained by automated size-based microfluidic sorting driven by artificial intelligence of culture expanded MSCs.

In an exemplary embodiment, the homogeneous population of MSCs is obtained by a process comprising the steps of:
a) culturing a heterogeneous population of MSCs in a culture medium for a period in a range of 20 to 35 days; b) staining the MSCs to obtain a heterogeneous population of stained MSCs; c) adding droplet encapsulation media, comprising a density gradient solution to the heterogenous population of stained MSCs, to obtain a population of microfluidic droplets, wherein each droplet preferably comprises a single MSC; d) providing the population of microfluidic droplets obtained from step (c) to a microfluidics device; and e) identifying and selecting a homogeneous population of MSCs of medium size in the range of 15 to 30 μm; wherein the step (e) further comprises: imaging the MSCs in the population of microfluidic droplets to record the size of the MSCs; and subjecting the population of microfluidic droplets to automated size-based sorting to obtain a population of homogeneous MSCs having medium size; and wherein the step (a) further comprises: reseeding the heterogeneous population of MSCs at a cell density in the range of 5000 to 11,000/cm² and expanding the heterogeneous population of MSCs up to passage 3, wherein the population of MSCs obtained from step (e) are viable MSCs, expressing MSC specific markers selected from CD 73, CD 90, and combinations thereof; and having increased expression of genes selected from the group consisting of COL12 A1 gene, IGFBP5 gene, THBS2 gene, GREM1 gene, CDH2 gene, and combinations thereof.

The term “medium-sized MSCs” as used herein refers to a homogeneous population of MSCs having a size in the range of 15 to 30 μm.

In an embodiment, the population of MSCs is of medium size in the range of 15 to 30 μm. In another embodiment, the population of MSCs is of medium size in the range of 17 to 22 μm.

In further embodiments, the medium-sized MSCs are characterized by the expression of certain genes that aid in their therapeutic potential. In an embodiment, medium-sized MSCs are characterized by the increased expression of genes selected from a group consisting of COL12A gene, IGFBP5 gene, THBS2 gene, GREM1 gene and combinations thereof.

In an embodiment, the MSCs exhibit increased expression of COL12A1 gene, IGFBP5 gene, THBS2 gene and GREM1 gene, as compared to the expression of GAPDH gene, wherein the increased expression of COL12A1 is in the range of 14 to 19 folds as compared to the expression of GAPDH gene; wherein the increased expression of IGFBP5 gene is in the range of 14 to 18 folds as compared to the expression of GAPDH gene; wherein the increased expression of THBS2 gene is in the range of 15 to 17 folds as compared to the expression of GAPDH gene; wherein the increased expression of GREM 1 gene is in the range of 18 to 21 folds as compared to the expression of GAPDH gene.

In an embodiment, the formulation comprises secretome of said MSCs.

The term “secretome” as used herein refers to the biologically active substances that are secreted by the MSCs and have the paracrine ability. The secretome is composed of cytokines, chemokines, growth factors, proteins, and extracellular vesicles. In an embodiment, the formulation comprises secretome of said MSCs comprising VEGF and IL-10.

The term “Vascular endothelial growth factor (VEGF)” as used herein refers to a family of polypeptides that includes VEGF-A, VEGF-B, VEGF-C, VEGF-D and placental growth factor (PlGF). VEGF shows paracrine and autocrine properties and can act intracellularly, secreting to the extracellular space, participating in the regulation of the cell-cycle and metabolism of cells. VEGF, an important angiogenic factor secreted by MSCs, promotes cell survival by inducing the expression of anti-apoptotic molecules such as Bcl-2.

The term “Interleukin-10 (IL-10)” as used herein refers to a type of cytokine that is found in the secretome of MSCs. IL-10 is characterized by its anti-inflammatory effect related to the induction of immune tolerance. It is an anti-inflammatory cytokine that inhibits the IL-2 and Interferon (IFN)-G. The IL-10 act as an inducer for immune tolerance on the dendritic cells. It has been established that IL-10 suppresses the functions of macrophages and neutrophils, inhibits the Th1 immune response, influences NF-κB synthesis and causes expression of anti-inflammatory molecules, such as protease inhibitors and IL-1 and TNFα antagonists.

The major function of IL-10 in induction of immune tolerance is its effect on the antigen presenting cells and particularly on the dendritic cells (DCs). IL-10 suppresses the secretion of pro-inflammatory cytokines (TNFα, IL-1, IL-6, IL-8, IL-12) by DCs and the expression of MHC II molecules, as well as co-stimulatory complex B7 on their surface. In parallel to that, IL-10 is capable of inducing anergy of T lymphocytes by directly inhibiting the phosphorylation of CD28. In that way, one of the basic immunosuppressive mechanisms is executed by IL-10 by inducing a tolerogenic type of dendritic cells with reduced HLA-II and B7 expression and by suppression of CD28 (the partner of B7) expression on the surface of the T lymphocytes. This “two sided” suppression of the second signal which is unconditionally needed for activation of the T lymphocytes induces a deep anergy in this cell population. Overall, IL-10 acts as a key immunoregulatory molecule, playing a crucial role in this “two-sided” suppression, leading to T cell anergy and preventing excessive immune responses that could damage healthy tissues.

In an embodiment, the formulation comprises secretome of said MSCs comprising VEGF in an amount in the range of 2050 to 2390 μg/ml, and IL-10 in an amount in the range of 1430 to 1690 ng/ml.

In an embodiment, the population of MSCs comprises less than 6% of early apoptotic cells; and less than 3% of late apoptotic cells.

In an embodiment, the MSCs are present in the range of 0.5×10⁶ cells/ml to 2×10⁶ cells/ml of the formulation, preferably 1.8×10⁶ cells/ml of the formulation.

In an embodiment, the homogeneous population of MSCs are present in a suspension comprising an excipient selected from DMEM, human serum albumin (HSA) or combinations thereof; preferably the excipient is DMEM and 20% HSA solution in a weight ratio in a range of 1:2 to 2:1, more preferably the excipient is DMEM and 20% HSA solution in a weight ratio of 1:1.

The term “carrier” as used herein refers to any known carrier, or adjuvants known to a person skilled in the art, and which can be used for preparing the formulation comprising the population of MSCs for therapeutic applications and is pharmaceutically acceptable.

In an embodiment, the carrier is in a weight percentage range of 0.5 to 2.5% of the formulation.

In an embodiment, the carrier is selected from serelaxin, Ringer's lactate solution, human serum albumin (HSA), heparin, dextran, hyaluronidase, or combinations thereof.

The term “serelaxin” as used herein refers to a recombinant form of human relaxin 2, a naturally occurring peptide. It exerts its effects by binding to one of two receptors, LGR7 and LGR8, to activate a G protein coupled receptor pathway and upregulates the vascular endothelin B receptor, vascular endothelin growth factor (VEGF). Serelaxin with an acceptable hemodynamic profile and no known infusion-related significant adverse drug events is used as one of the components of carrier solution of the present disclosure.

In an embodiment, the serelaxin is a 0.5 to 5 ng/ml concentration solution. In another embodiment, the serelaxin is a 0.75 to 3 ng/ml concentration solution. In yet another embodiment, the serelaxin is a 0.9 to 1.5 ng/ml concentration solution.

The term “Ringer's lactate (RL)” as used herein refers to a mixture of sodium ions (Na⁺), chloride ions (Cl⁻), potassium ions (K⁺), calcium ions (Ca²⁺), and lactate ions mimicking the extracellular fluid in the human body. Several mechanisms are found to support the protective functions of RL on cell viability, which are linked to the extracellular concentrations of Na⁺, K⁺, and Ca²⁺ ions.

In an embodiment, Ringer's lactate solution comprises salts of sodium lactate, sodium chloride, potassium chloride, and calcium chloride in water.

In an embodiment, Ringer's lactate solution comprises 25 to 30 mM of sodium lactate, 100 to 105 mM of sodium chloride, and 3.5 to 4.5 mM of potassium chloride and 1.5 to 2 mM of calcium chloride. In another embodiment, Ringer's lactate solution comprises 26 to 29 mM of sodium lactate, 101 to 103 mM of sodium chloride, and 3.8 to 4.1 mM of potassium chloride and 1.5 to 2 mM of calcium chloride.

The Ringer's lactate solution may be prepared by methods generally known in the art. In an exemplary embodiment, the Ringer's lactate solution is prepared by mixing predetermined amounts of sodium lactate, sodium chloride, potassium chloride, and calcium chloride in water.

In an embodiment, the Ringer's lactate solution has an osmolarity in the range of 270 mOsm/L to 275 mOsm/L.

The term “Human Serum Albumin (HSA)” as used herein refers to a lipid peroxidation inhibitor or an emulsifying substance to protect lipids against oxidation. Further, HSA plays a significant role in the membrane structure stabilization and reduces cell damage caused by osmotic stress and has anti-oxidative activity, so it helps to maintain osmolarity of the formulation.

In an embodiment, the HSA is a 1 to 20% solution of HSA. In another embodiment, the HSA is a 10 to 20% solution of HSA.

The term “heparin” as used herein refers to an anticoagulant that prevents the formation of blood clots. Heparin prevents the blood clot formation while delivering the MSCs and also improves the effectiveness of cell therapy.

In an embodiment, heparin is in a concentration range of 500 to 2000 U/ml. In another embodiment, heparin is in a concentration range of 750 to 1500 U/ml.

The term “dextran” as used herein refers to a polysaccharide composed of glucose. It is made from natural sources of glucose, and this makes it generally well-tolerated by the body and reduces the risk of immune reactions compared to synthetic materials when used in a therapeutic formulations. Dextran occurs in various molecular weights, such as dextran 40 (40 kDa) and dextran 70 (70 kDa), which are both commonly used as carriers. Dextran 40, when used along with cells can be designed for slow release. This allows for a more gradual delivery of the cells to the target site, potentially improving their survival and therapeutic effect. Also, dextran can be chemically modified to target specific tissues. This could be useful for directing the delivered cells to a particular area of the body for treatment.

In an embodiment, dextran is a 0.5% to 19% concentration solution. In another embodiment, dextran is a 1% to 5% concentration solution.

In an embodiment, the formulation further comprises hyaluronidase.

The term “hyaluronidase” as used herein refers an enzyme that breaks down hyaluronic acid (HA). HA is a major component of the extracellular matrix of subcutaneous tissue. Hyaluronidase specifically cleaves the glycosidic bonds within the hyaluronic acid molecule, essentially breaking it down. This breakdown creates temporary channels within the ECM, allowing other injected fluids and medications to diffuse more readily throughout the tissue. Thus, hyaluronidase increases tissue permeability when used in a medication, allowing the injected medication to spread more easily, and be absorbed faster into the bloodstream. With quicker absorption, the medication reaches its target site and starts working sooner. This can be particularly beneficial for drugs where a rapid response is critical. Hyaluronidase can facilitate the administration of larger volumes of medication subcutaneously. Normally, injecting a large volume into a localized area can be uncomfortable or cause pooling of the medication. Hyaluronidase helps the medication disperse more readily, reducing discomfort and improving delivery. Also, certain medications can irritate or damage tissues if they pool at the injection site. By promoting faster absorption, hyaluronidase can minimize this risk and improve patient comfort.

In an embodiment, hyaluronidase is in a concentration range of 500 to 5000 IU/ml of the carrier. In an embodiment, hyaluronidase is in a concentration of 1500 units to 3000 units per ml of the carrier.

In an embodiment, the serelaxin is a 0.5 to 5 ng/ml concentration solution; the Ringer's lactate solution comprises 25 to 30 mM of sodium lactate, 100 to 105 mM of sodium chloride, and 3.5 to 4.5 mM of potassium chloride and 1.5 to 2 mM of calcium chloride; and HSA is a 1 to 20% concentration solution; dextran is a 0.5% to 19% concentration solution; heparin is in a concentration range of 500 to 2000 U/ml; and hyaluronidase is in a concentration range of 500 to 5000 U/ml.

In an embodiment, the formulation comprises additives. The term “additive” as used herein refers to substances that are optionally provided in the formulation to improve its stability.

In an embodiment, the formulation comprises additives selected from DMEM medium, saline solution, phosphate buffered saine (PBS) buffer, Hank's balanced salt solution (HBSS), human plasma, plasma lysate, or mixtures thereof.

In an embodiment, the formulation has a pH in the range of 6.4 to 6.6.

In an embodiment, the formulation has an osmolarity in the range of 275 to 310 mOsm/L.

In an embodiment, the percentage cell recovery of said MSCs in the formulation estimated after 0 to 24 hours is in the range of 99 to 80% and a percentage cell viability of said MSCs in the formulation estimated after 0 to 24 hours is in the range of 99.8% to 80%.

Embodiments herein include a method of preparing the formulation.

In an embodiment, the method for preparing the formulation comprises: mixing the plurality of MSCs with the carrier to obtain the formulation.

In an embodiment, the method further comprises the addition of one or more additives.

Embodiments herein further include a method of inducing tissue regeneration.

In an embodiment, there is provided a method of inducing tissue regeneration comprising: administering to a tissue in need thereof the formulation as disclosed herein or obtained by the method as disclosed herein; wherein the tissue is selected from epithelial tissue; connective tissue like bone tissue, cartilage tissue, or elastic tissue; muscle tissue, or nervous tissue.

Embodiments herein further include a method of treating autoimmune or fibrotic disease in a subject.

The term “subject”, as used herein, refers to mammals, e.g., human, and non-human mammals. Examples of non-human animals include non-human primates, dogs, cats, cattle, horses, sheep, pigs, goats, rabbits, mice, rats, hamsters, guinea pigs and etc. Unless otherwise noted, the terms “patient” or “subject” are used herein interchangeably. Preferably, the subject is human.

In an embodiment, there is provided a method of treating a disease in a subject, comprising administering the formulation as disclosed herein or obtained by the method as disclosed herein to the subject.

In an embodiment, the formulation is administered via the intravenous, intramuscular, intraosseous, subcutaneous, intraplantar, or intraarticular route.

In an embodiment, there is provided a use of the formulation as disclosed herein in the manufacture of a medicament for treating autoimmune or fibrotic disease in a subject.

In an embodiment, the auto immune disease or fibrotic disease is selected from the group of consisting of Acromegaly, Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barre Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, *Juvenile polymyositis/Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndrome (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Rheumatoid Arthritis (RA), Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis (TM), Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, ureteric stricture, eye fibrosis, heart fibrosis, hepatic fibrosis, intestinal fibrosis, lung fibrosis, Pancreas fibrosis, renal fibrosis, and skin fibrosis.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Materials

Components of ringer lactate (274 mOsm/L) procured commercially is as follows:

$$1. Sodium Lactate ( C 3 H 5 NaO 3 ) - 1.55 g / 500 mL$$

$$2. Sodium Chloride ( NaCl ) - 3. g / 500 mL$$

$$3. Potassium Chloride ( KCL ) - 1.15 g / 500 mL$$

$$4. Calcium Chloride ( CaCl 2 \cdot 2 H 2 O ) - 0.1 g / 500 mL$$

$$5. Water for injection - 500 mL$$

$$6. Na + 130.3 mEq / L$$

$$7. K + 4 mEq / L$$

$$8. Lactate ( HCO 3 - ) - 27.7 mEq / L$$

$$9. Cl - 109.4 mEq / L$$

$$10. Ca \text{++} 2.7 mEq / L$$

### Example 1: Preparation of Formulation

Mesenchymal stem cells from umbilical cord tissues (UCT) from single or multiple donors were isolated and expanded in vitro up to passage 3 (P3) by using 2D and 3D culture system. In 2D culture system culture flasks were used, while in 3D culture system such as in a continuous feedback system, a microcarrier which has a porous membrane, hollow fibre bioreactor of pore size of 80 μm, and enables scalable cell culture was used. Microfluidic system sorting of these expanded cells at P3 stage was performed to obtained medium sized MSCs of 15 to 30 μm, and cell banking of these medium size sorted cells was carried out. Freshly sorted UCMSCs as well as some cryopreserved UCMSC (using 20% Human serum albumin (HSA) with 10% DMSO below −150° C. till thawing) were used for analysis of delivery system by using different carrier solution. In cryopreservation, after thawing, the mesenchymal stem cells were suspended in DMEM+20% HSA (1:1) as excipient. The excipient remains same for fresh or cryopreserved product. This final product was resuspended into five types of carrier in different formulations of isotonic electrolyte solutions, namely 0.9% Sodium chloride (NaCl) and HSA (0.4%) (F1); Ringer Lactate and HSA (0.4%) (F2); Ringer lactate and 0.4% HSA and Dextran 40 (1%) (F3); Ringer lactate, 1% HSA and Dextran 40 (1%)+Heparin (1000 U/ml) (F4); Ringer lactate, 1% HSA, Dextran 40 (1%), Heparin (1000 U/ml), and Serelaxin (F5); and hyaluronidase (F6) by maintaining temperature at 15-28° C.

The dose designed and established for humans was 1 to 5 million cells/kg, preferably 5 million cells/kg body weight of human. Considering this value, the final concentration of MSCs in each carrier solution was 0.7×10⁶ MSCs/ml in a final volume of 500 ml to obtain the formulation. The cell count was taken as soon as the final product are suspended in carrier solution for 0 h readings. The formulation (˜100 ml) was collected from collection port using syringe and needle under aseptic conditions in 50 ml centrifuge tubes (2-3 tubes) at a speed of 5 ml/minute. Before collection the formulation was mixed well. The collected formulation was then centrifuged at 1300 rpm for 5-10 minutes at room temperature to obtain a cell suspension (cell pellet suspended in DMEM+20% HSA (1:1)) and a cell supernatant. Similarly, cell suspension and supernatants were obtained, and analysis performed after storing the formulation for 6 h, 12 h and 24 h; or 3 h, 6 h and 12 h.

Cell recoveries were calculated from the cell suspension at same time points, such as 0 h, 6 h, 12 h and 24 h or 0 h, 3 h, 6 h and 12 h using an automated counter. The cell recovery percentage was calculated using the below formulae:

$$Cell recovery \% = ( Initial cell count / Final cell count ) \times 100$$

Cell Viability of the cells were determined by staining 100 μl cells with 10 μl of 7AAD stain in a tube. 100 μl of unstained cells were taken in another tube as control.

Both the tubes were incubated for 10 min. After incubation 400 μl of PBS buffer was added and the tubes were vortexed gently before loading. The samples were acquired on flow cytometry and analysed to determine the percentage of viable and non-viable cells.

Cell surface positive markers and negative markers were estimated at different time points such as 0 h, 6 h, 12 h, and 24 h from cell suspension using AAD-flow cytometer. Briefly, cells were stained with 5 μl cell surface marker specific (Positive and Negative) antibody conjugated with respective fluorochromes (PE/FITC/PerCP/APC) in a tube. The tube was vortexed gently to mix and incubated for 30 minutes in the dark at 2-80° C. Simultaneously a tube of unstained cells was used as control. 500 μl of PBS was added to both stained and unstained tube of cells. The suspension was centrifuged at 1500 rpm for 5 mins, then the supernatant was discarded, and the pellet was resuspended in 500 μl of PBS. The tubes were gently vortexed before loading. The samples were acquired on the flow cytometer within 1 hour or until then the tubes on were placed on wet ice in the dark. After the acquisition, the graphs were analysed to determine the positive and negative markers expression on the MSCs.

Annexin V Analysis was performed to determine the apoptotic status of cells. Briefly, 100 μl of cells were stained with 5 μl of Annexin V in a tube. 100 μl of unstained cells were taken in another tube as a control. The tubes were incubated for 10 min at room temperature. After incubation, 195 μl 1× Buffer was added to the tubes, then centrifuged at 1000 rpm for 2-3 min. The supernatant was discarded, and 10 μl of PI was added to the cell pellet and mixed well. Further, 190 μl of 1× buffer was added, and vortexed gently to mix. The samples were acquired on flow cytometry.

After acquisition, analysis was performed to determine healthy cells, early apoptotic cells, late apoptotic cells, and dead cells population in the sample.

Secretome analysis (IL-10 and VEGF analysis): Standard dilutions of the were prepared. Microwell strips were washed twice with wash buffer, before adding 100 μl of these standard dilutions in the microwell strips. 100 μl of sample diluent, in duplicate, was added to the blank wells. 50 μL of sample diluent was added to sample wells. Then 50 μl sample in duplicate was added to designated sample wells. 50 μl of Biotin-Conjugate was added to all wells. The microwell strips were covered and incubated for 2 hours at room temperature (18° C. to 25° C.). The microwell strips were emptied and washed 3 times with Wash Buffer. 100 μL of diluted Streptavidin-HRP was added to all wells. The microwell strips were covered and incubated for 1 h at room temperature (18° C. to 25° C.). The microwell strips were emptied and washed 3 times with Wash Buffer. 100 μL of TMB Substrate Solution was added to all wells. The microwell strips were incubated for about 10 minutes at room temperature (18° C. to 25° C.). Then 100 μl of Stop Solution was added to all wells. Blank microwell reader and colour intensity was measured at 450 nm.

pH, and osmolarity were analysed using the cell supernatant with a pH meter, and osmometer, respectively.

Gene expression studies was carried out using the formulation at 0 h- and 24 h-time point only by RT-PCR. Briefly, RNA was isolated from target MSCs cells. mRNA was reverse transcribed to cDNA. The modified gene-specific PCR primers for the required genes (COL12 A1; IGFBP5; THBS2; GREM1) were used to amplify a segment of the cDNA of interest, following the reaction in real time; and the initial concentration of the selected transcript in the MSCs was calculated from the exponential phase of the reaction. The primers used are listed below:

COL12 A1 Forward:
CAGTGCCTGTAGTCAGCCTGAA
COL12 A1 Reverse:
GGTCTTGTTGGCTCTGTGTCCT
IGFBP5 Forward:
CGTGCTGTGTACCTGCCCAATT
IGFBP5 Reverse:
ACTTGTCCACGCACCAGCAGAT
THSB2 Forward:
CAGTCTGAGCAAGTGTGACACC
THSB2 Reverse:
TTGCAGAGACGGATGCGTGTGA
GREM1 Forward:
TCATCAACCGCTTCTGTTACGGC
GREM1 Reverse:
CAGAAGGAGCAGGACTGAAAGG

TABLE 1
Analysis of homogeneous medium size 1 × 10⁶ UC-MSCs

| Parameters | Result |
|---|---|
| Cell Viability (%) | 98.90 |

Annexin V

| | |
|---|---|
| Early Apoptotic cells (%) | 0.18 |
| Late Apoptotic cells (%) | 0.00 |

CELL SURFACE MARKERS (%)
Positive Markers

| | |
|---|---|
| CD90+ | 99.20 |
| CD73+ | 97.85 |
| CD105+ | 98.60 |

Negative marker

| | |
|---|---|
| CD34− | 0.22 |
| CD45− | 0.07 |
| HLADR− | 0.011 |

Potency (Secretomes estimation)

| | |
|---|---|
| IL10 ng/ml | 1430-1690 |
| VEGF pg/ml | 2050-2390 |

Gene expression (Fold expression) *

| | |
|---|---|
| COL12 A1 | 19.5 |
| IGFBP5 | 18.22 |
| THBS2 | 17.80 |
| GREM1 | 21.25 |

### Table Descriptions

*Data represented as fold expression when compared with GAPDH gene.

One million cells comprising of 9.92×10⁵ CD90 cells. 9.78×10⁵ CD73 cells and 9.86×10⁵ CD105 cells.

One million cells comprising of 9.89×10⁵ viable cells with only 1800 early apoptotic cells and without any late apoptotic cells. One million homogeneous medium size MSCs population comprising of 1430-1690 μg of IL 10 Secretomes.

One million homogeneous medium size MSCs population comprising of 2050-2390 μg of VEGF Secretomes.

One million homogeneous medium size MSCs showing gene expression of 19.5-fold COL12 A1, 18.22-fold of IGFBP5, 17.80-fold of THBS2 and 21.25-fold of GREM1.

### Example 2: F1-Formulation Comprising NaCl (0.9%) and HSA (0.4%) as Carrier

Result suggested that HUC-MSCs could be ideally stored in 0.9% NaCl and 0.4% HSA at 15-28° C. for up to 6 h with a maximum viability of 82%. NaCl-based media did not support the viability of UC-MSC at room temperature. A significant decrease in cell viability in NaCl and HSA solution was observed. It was also noted that other biological characteristics, such as immunophenotype, and immunosuppressive were affected (Table 2).

TABLE 2
Evaluation of the effects of carrier comprising
NaCl (0.9%) and HSA (0.4%) on UC-MSC

| UC-MSC quality | Time interval (In Hours) | | | |
|---|---|---|---|---|
| check parameters | 0 h | 6 h | 12 h | 24 h |
| Cell recovery (%) | 99 | 83 | 71 | 65 |
| Cell Viability (%) | 99.69 | 82.22 | 69.34 | 54.51 |

Annexin V

| | | | | |
|---|---|---|---|---|
| Early Apoptotic cells (%) | 2.87 | 9.62 | 18.87 | 27.00 |
| Late Apoptotic cells (%) | 0.00 | 1.030 | 2.006 | 3.450 |
| pH | 6.5 | 6.4 | 6.1 | 6.0 |
| OSMOLARITY | 273 | 250 | 241 | 212 |
| (mOsm/L) | | | | |

CELL SURFACE MARKERS (%)
Positive Markers

| | | | | |
|---|---|---|---|---|
| CD90+ | 99.860 | 73.821 | 61.851 | 53.153 |
| CD73+ | 96.541 | 76.541 | 64.321 | 56.121 |
| CD105+ | 96.541 | 76.251 | 70.111 | 52.111 |

Negative marker

| | | | | |
|---|---|---|---|---|
| CD34− | 0.27 | 0.22 | 0.36 | 0.12 |
| CD45− | 0.08 | 0.001 | 0.02 | 0.15 |
| HLADR− | 0.014 | 0.03 | 0.01 | 0.05 |

Potency (Secretomes estimation)

| | | | | |
|---|---|---|---|---|
| IL10 ng/ml | 955 | 968 | 1026 | 973 |
| VEGF pg/ml | 1653 | 1700 | 1795 | 1698 |

Gene expression (Fold expression) *

| | | | | |
|---|---|---|---|---|
| COL12 A1 | 17.80 | — | — | 11.56 |
| IGFBP5 | 16.90 | — | — | 10.33 |
| THBS2 | 15.40 | — | — | 10.98 |
| GREM1 | 20.50 | — | — | 14.97 |

* Data represented as fold expression when compared with GAPDH gene.

### Example 3: F2-Formulation Comprising Ringer Lactate+HSA (0.4%) as Carrier

The concentration of 0.4% HSA was chosen because it has been used intensively as a supplement for preserving immune cells and is effective in maintaining the high quality of these cells before administration as a suspension solution. Ringer's lactate solution (RL), also known as sodium lactate solution, was conceived as an injectable solution for medical use. The osmolarity of RL is 275.52 mOsm/L, which is almost in the physiological osmotic range (280-310 mOsmol/L). It is an isotonic fluid and contains some extra salts (potassium, calcium, chloride, lactate), which support cells more efficiently compared with 0.9% saline. The supplementation of Ringer lactate with 0.4% HSA showed improvement in viability of MSCs (Table 3) at 12 h and 24 h as compared to the use of 0.9% NaCl and 0.4% HSA as carrier.

TABLE 3
Evaluation of the effects of carrier
Ringer Lactate + 0.4% HSA on UC-MSC

| UC-MSC quality | Time interval | | | |
|---|---|---|---|---|
| check parameters | 0 h | 6 h | 12 h | 24 h |
| Cell recovery (%) | 99 | 86 | 81 | 78 |
| Cell Viability (%) | 96.41 | 93.01 | 88.33 | 76.46 |

Annexin V

| | | | | |
|---|---|---|---|---|
| Early Apoptotic cells (%) | 3.17 | 9.04 | 10.21 | 11.41 |
| Late Apoptotic cells (%) | 0.00 | 1.004 | 2.659 | 3.964 |
| pH | 6.5 | 6.3 | 6.0 | 5.9 |
| Osmolarity (mOsm/L) | 275 | 270 | 266 | 261 |

CELL SURFACE MARKERS (%)
Positive Markers

| | | | | |
|---|---|---|---|---|
| CD90+ | 97.28 | 92.26 | 87.16 | 81.36 |
| CD73+ | 98.58 | 93.52 | 87.16 | 82.65 |
| CD105+ | 99.54 | 91.36 | 86.36 | 81.35 |

Negative markers

| | | | | |
|---|---|---|---|---|
| CD34− | 0.15 | 0.37 | 0.76 | 0.17 |
| CD45− | 0.05 | 0.036 | 0.07 | 0.09 |
| HLADR− | 0.02 | 0.07 | 0.11 | 0.27 |

Potency (Secretomes estimation)

| | | | | |
|---|---|---|---|---|
| IL10 (ng/ml) | 1026 | 1123 | 1189 | 1010 |
| VEGF | 1795 | 1860 | 1820 | 1077 |
| (pg/ml) | | | | |

Gene expression (Fold expression)*

| | | | | |
|---|---|---|---|---|
| COL12 A1 | 17.80 | — | — | 13.09 |
| IGFBP5 | 16.90 | — | — | 14.66 |
| THBS2 | 15.40 | — | — | 13.39 |
| GREM1 | 20.50 | — | — | 17.66 |

*Data represented as fold expression when compared with GAPDH gene.

### Example 4: F3-Formulation Comprising Ringer Lactate+0.4% HSA+Dextran 40 (1%) as Carrier

RL along with 0.4% HSA and Dextran 40 (1%) supported the survivability of MSCs derived from umbilical cord tissue for up to 24 h (Table 4).

TABLE 4
Evaluation of the effects of carrier Ringer lactate +
0.4% HSA + Dextran 40 (1%) on UC-MSC

| UC-MSC quality | Time interval | | | |
|---|---|---|---|---|
| check parameters | 0 h | 6 h | 12 h | 24 h |
| Cell recovery (%) | 99 | 93 | 85 | 79 |
| Cell Viability (%) | 95.53 | 94.61 | 91.77 | 89.13 |

Annexin V

| | | | | |
|---|---|---|---|---|
| Early Apoptotic cells (%) | 2.48 | 4.51 | 6.38 | 7.05 |
| Late Apoptotic cells (%) | 0.00 | 1.364 | 3.628 | 5.624 |
| pH | 6.5 | 6.4 | 6.4 | 6.3 |
| Osmolarity (mOsm/L) | 275 | 270 | 266 | 26 |

CELL SURFACE MARKERS (%)
Positive Markers (%)

| | | | | |
|---|---|---|---|---|
| CD90+ | 99.26 | 97.12 | 89.12 | 84.12 |
| CD73+ | 97.56 | 93.35 | 87.58 | 82.35 |
| CD105+ | 98.56 | 94.65 | 86.35 | 81.65 |

Negative marker (%)

| | | | | |
|---|---|---|---|---|
| CD34− | 0.13 | 0.36 | 0.45 | 0.98 |
| CD45− | 0.33 | 0.64 | 0.07 | 0.06 |
| HLADR− | 0.38 | 0.06 | 0.31 | 0.03 |

Potency (Secretomes estimation) pg/ml

| | | | | |
|---|---|---|---|---|
| IL10 (Ng/ml) | 1026 | 1120 | 1236 | 1004 |
| VEGF (pg/ml) | 1795 | 1801 | 1823 | 1705 |

Gene expression (Fold expression)*

| | | | | |
|---|---|---|---|---|
| COL12 A1 | 17.80 | — | — | 12.68 |
| IGFBP5 | 16.90 | — | — | 12.04 |
| THBS2 | 15.40 | — | — | 12.10 |
| GREM1 | 20.50 | — | — | 17.69 |

*Data represents as fold Expression when compared with GAPDH gene.

### Example 5: F4-Formulation Comprising Ringer Lactate+1% HSA+Dextran 40 (1%)+Heparin (1000 U/Ml) as Carrier

HSA works like bovine serum albumin (BSA), as a lipid peroxidation inhibitor or an emulsifying substance to protect lipids against oxidation, plays a significant role in the membrane structure stabilization and reduces damage caused by osmotic stress. It has anti-oxidative activity and so it helps to maintain osmolarity. RL with 1% HSA, dextran 40 (1%) and heparin (1000 U/ml) supported the survivability of MSCs (93.70%) derived from umbilical cord tissue for up to 24 h (Table 5).

TABLE 5
Evaluation of the effects of carrier Ringer lactate + 1%
HSA + Dextran 40 (1%) + Heparin (1000 Units) on UC-MSC

| UC-MSC quality | Time interval | | | |
|---|---|---|---|---|
| check parameters | 0 h | 6 h | 12 h | 24 h |
| Cell recovery (%) | 99 | 96 | 94 | 91 |
| Cell Viability (%) | 99.20 | 97.40 | 95.15 | 93.70 |

Annexin V

| | | | | |
|---|---|---|---|---|
| Early Apoptotic cells (%) | 0.85 | 1.91 | 3.23 | 3.62 |
| Late Apoptotic cells (%) | 0.00 | 0.964 | 2.635 | 3.462 |
| pH | 6.5 | 6.5 | 6.4 | 6.3 |
| Osmolarity (mOsm/L) | 275 | 273 | 270 | 268 |

CELL SURFACE MARKERS (%)
Positive Markers (%)

| | | | | |
|---|---|---|---|---|
| CD90+ | 98.28 | 92.68 | 85.64 | 81.78 |
| CD73+ | 99.12 | 93.62 | 89.46 | 83.68 |
| CD105+ | 97.99 | 94.99 | 88.89 | 83.35 |

Negative marker (%)

| | | | | |
|---|---|---|---|---|
| CD34− | 0.35 | 0.98 | 0.65 | 0.15 |
| CD45− | 0.06 | 0.01 | 0.13 | 0.30 |
| HLADR− | 0.03 | 0.008 | 0.13 | 0.95 |

Potency (Secretomes estimation)

| | | | | |
|---|---|---|---|---|
| IL10 (ng/ml) | 1026 | 1041 | 1068 | 998 |
| VEGF (pg/ml) | 1795 | 1806 | 1834 | 1712 |

Gene expression (Fold expression)*

| | | | | |
|---|---|---|---|---|
| COL12 A1 | 17.80 | — | — | 14.73 |
| IGFBP5 | 16.90 | — | — | 13.99 |
| THBS2 | 15.40 | — | — | 13.01 |
| GREM1 | 20.50 | — | — | 17.47 |

*Data represents as fold expressions when compared with GAPDH gene.

### Example 6: F5-Formulation Comprising Ringer Lactate+1% Has+Dextran 40 (1%)+Heparin (1000 U/ml)+Serelaxin as Carrier

Results revealed that all tested UCMSCs steadily maintained CD73 and CD90 expression at 0 h and 24 h storage at RT in the tested media, the expression was higher than 85%. The expression of negative markers, including CD19, CD34, CD45 and HLA-DR remained below 10% regardless of storage media and storage duration.

Prolong exposure of MSCs in serum free medium have showed increase in secretomes. Notably, the preservation conditions directly altered the secretory profiles of MSCs from all the tested sources, and their behaviour in response to the surrounding environment was also distinct. This was proved by the increasing concentration of total protein concentration after 24 h. Carrier solution comprising Ringer lactate, 1% HSA, Dextran 40 (1%), Heparin (1000 U/ml) and Serelaxin (1 ng/ml), which gives better results than other carriers used in the study up to 24 h.

The results indicated that transport/storage of clinical-grade UC-MSCs successfully preserved viability rates above the allowable limits, stimulatory factors, pH, osmolarity and surface marker expression at 15-28° C. temperatures, preferably in RL-based media supplemented with 1% HSA, Dextran 40 (1%), Heparin (1000 U/ml) and Serelaxin (1 ng/ml).

In the gene expression studies for different genes such as COL 12A1, IGFBP5, THBS2 and GREM1, carrier solution having Ringer lactate 1% HSA, Dextran40 (1%), Heparin (1000 U/ml) and Serelaxin (Ing/ml) was found better when compared with other carrier solutions, The gene expression was found more in this carrier as compared with others (Table 6).

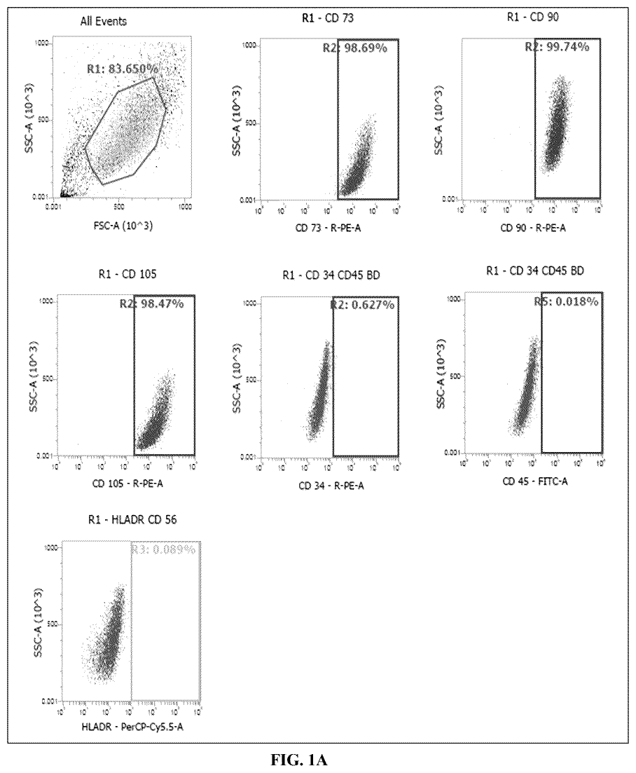

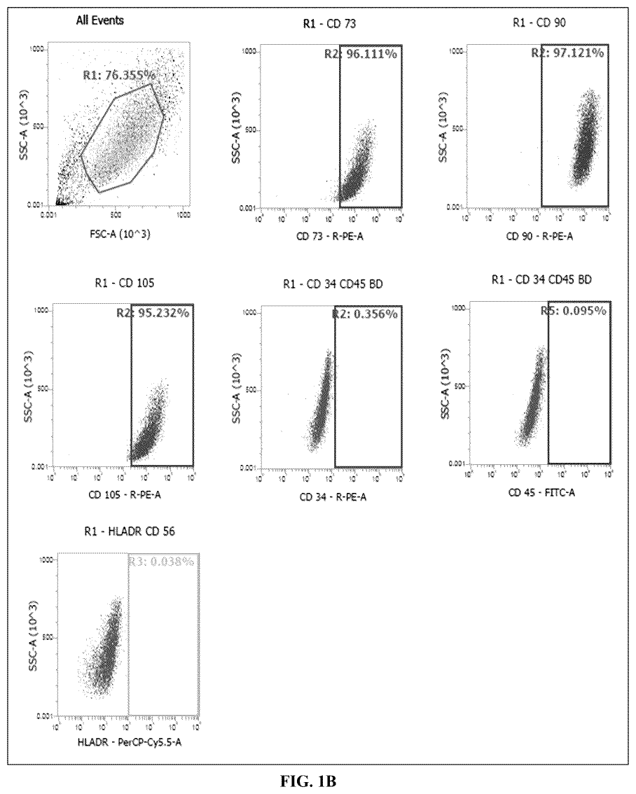

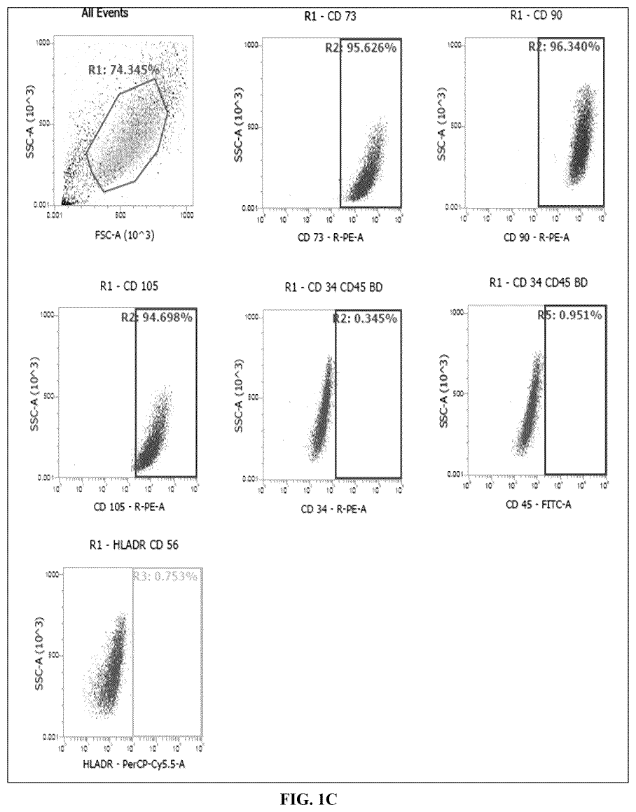

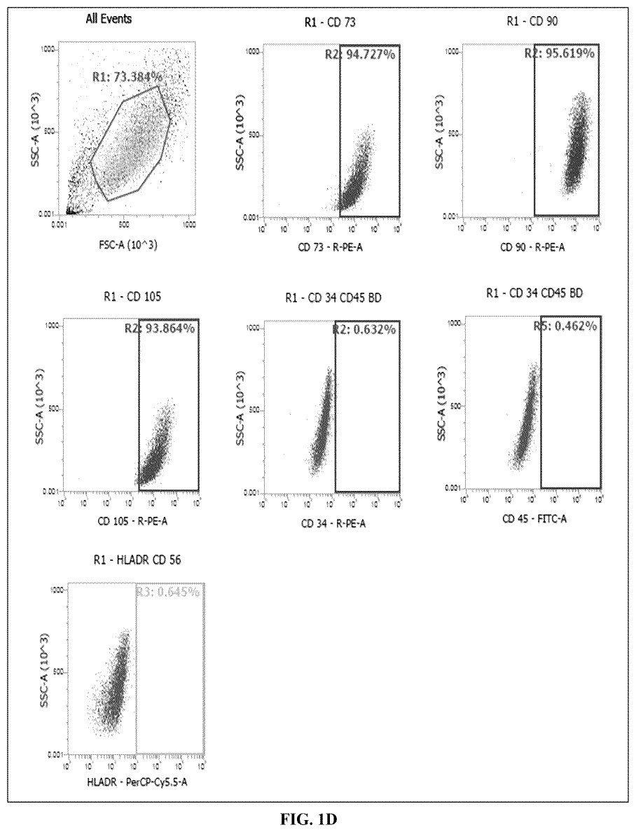

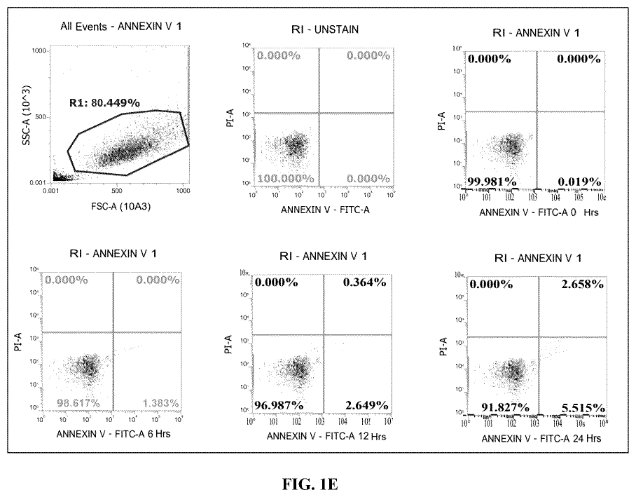

TABLE 6
Evaluation of the effects of carrier Ringer Lactate
(RL) 1% HSA, Dextran 40 (1%), Heparin (1000 U/ml)
and Serelaxin (1 ng/ml) on UC-MSC (FIG. 1A-1E)

| UC-MSC quality | Time Interval | | | |
|---|---|---|---|---|
| check parameters | 0 h | 6 h | 12 h | 24 h |
| Cell recovery (%) | 99 | 99 | 98 | 97 |
| Cell Viability (%) | 99.78 | 99.01 | 98.66 | 97.54 |

Annexin V

| | | | | |
|---|---|---|---|---|
| Early Apoptotic cells (%) | 0.019 | 1.38 | 2.65 | 5.51 |
| Late Apoptotic cells (%) | 0.00 | 0.00 | 0.364 | 2.658 |
| pH | 6.5 | 6.5 | 6.5 | 6.4 |
| Osmolarity (mOsm/L) | 275 | 280 | 282 | 310 |

CELL SURFACE MARKERS (%)
Positive Markers (%)

| | | | | |
|---|---|---|---|---|
| CD90+ | 99.74 | 97.12 | 96.340 | 95.619 |
| CD73+ | 98.69 | 96.11 | 95.626 | 94.727 |
| CD105+ | 98.47 | 95.23 | 94.698 | 93.864 |

Negative marker (%)

| | | | | |
|---|---|---|---|---|
| CD34− | 0.627 | 0.356 | 0.345 | 0.632 |
| CD45− | 0.018 | 0.095 | 0.951 | 0.462 |
| HLADR− | 0.089 | 0.038 | 0.753 | 0.645 |

Potency (Secretomes estimation)

| | | | | |
|---|---|---|---|---|
| IL10 (ng/ml) | 1026 | 1039 | 1169 | 1002 |
| VEGF (pg/ml) | 1795 | 1873 | 1888 | 1701 |

Gene Expression (Fold expression)

| | | | | |
|---|---|---|---|---|
| COL12 A1 | 17.80 | — | — | 16.40 |
| IGFBP5 | 16.90 | — | — | 15.50 |
| THBS2 | 15.40 | — | — | 15.20 |
| GREM1 | 20.50 | — | — | 18.25 |

*Data represented as fold expression when compared with GAPDH gene.

### Example 7: F6-Formulation Comprising Hyaluronidase as Carrier

Human hyaluronidase enzyme facilitates the subcutaneous delivery of the formulation when used along or before with hyaluronidase enzyme. This enzyme when administered locally, it depolymerizes hyaluronic acid in the subcutaneous space, temporarily removing the barrier to subcutaneous administration of relatively large volumes. The application of hyaluronidase enzyme to the existing subcutaneous drug formulation can optimize dosing and administration, remove the volume limitations associated with conventional subcutaneous delivery, and also enable reformulation of existing intravenous (IV) drugs for rapid subcutaneous delivery.

The required dose of hyaluronidase enzyme is 2000 IU/ml with the formulation of the present disclosure. This hyaluronidase enzyme shall be co-infused with the formulation or infused at same site before infusion/injection of the formulation.

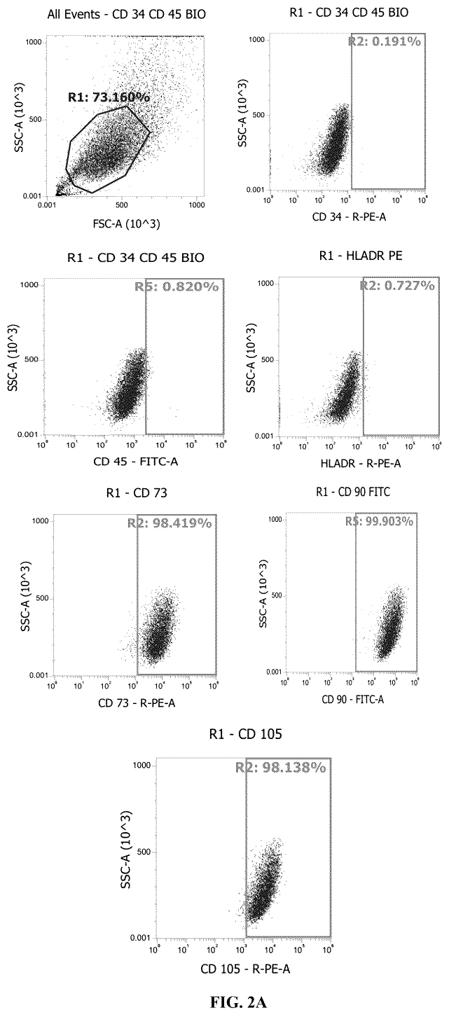

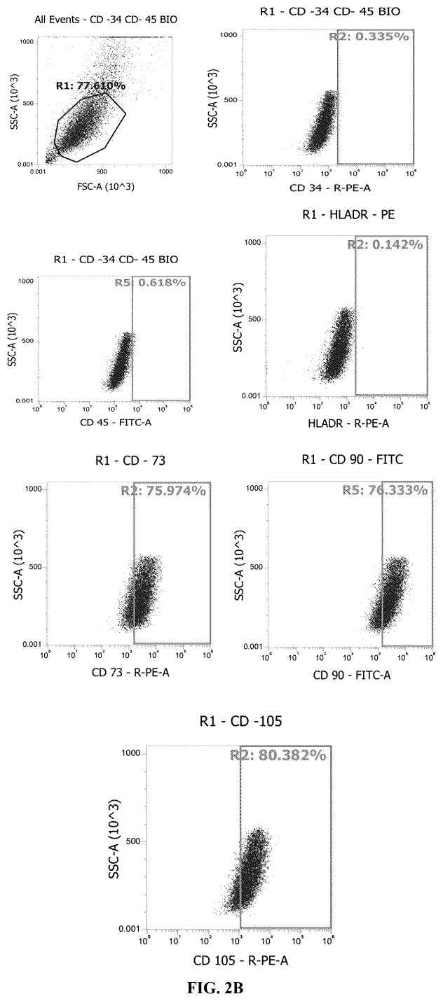

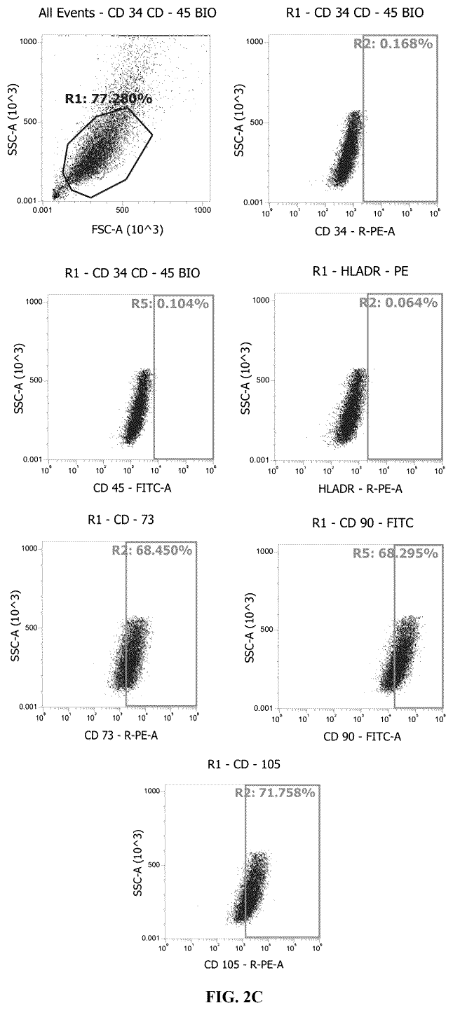

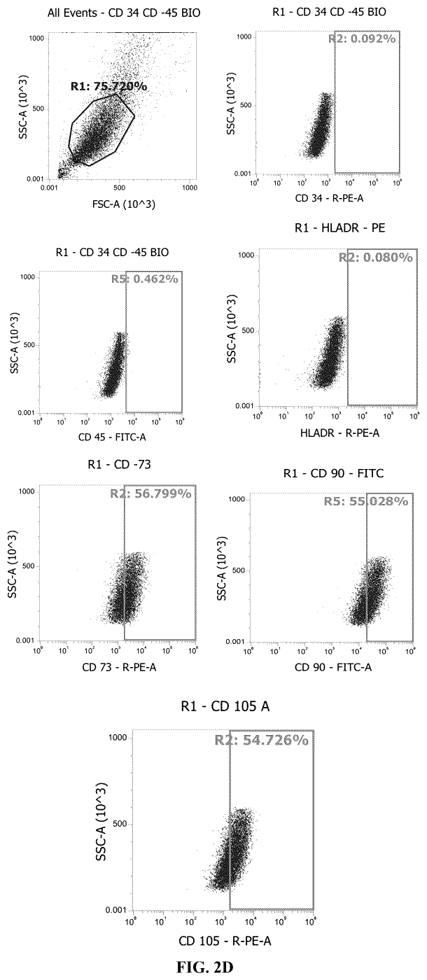

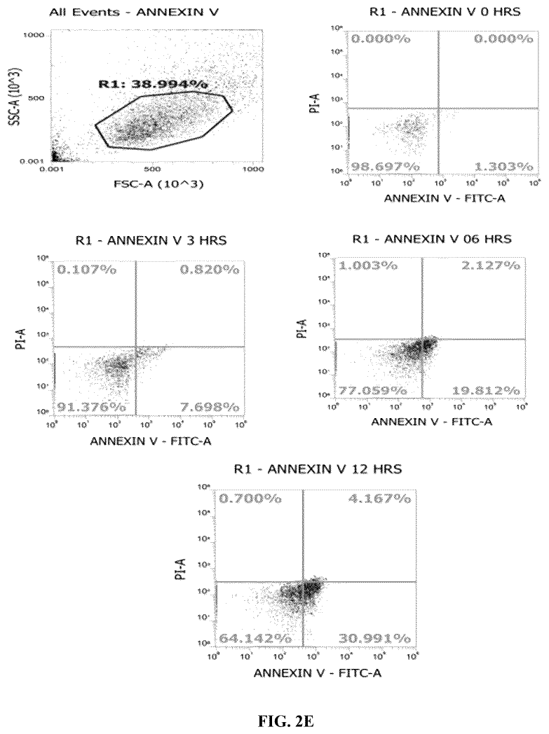

TABLE 7
Evaluation of the effect of carrier, Hyaluronidase
(2000 IU/ml) on UC-MSCs (FIG. 2A-2E)

| UC-MSC quality | Time interval (In Hours) | | | |
|---|---|---|---|---|
| check parameters | 0 h | 3 h | 6 h | 12 h |
| Cell recovery (%) | 98.10 | 81.20 | 70.15 | 64.35 |
| Cell Viability (%) | 99.18 | 84.14 | 67.46 | 53.41 |

Annexin V

| | | | | |
|---|---|---|---|---|
| Early Apoptotic cells (%) | 1.30 | 7.69 | 19.81 | 30.99 |
| Late Apoptotic cells (%) | 0 | 0.82 | 2.12 | 4.16 |
| pH | 6.6 | 6.5 | 6.1 | 6.0 |
| OSMOLARITY | 271 | 253 | 244 | 210 |
| (mOsm/L) | | | | |

CELL SURFACE MARKERS (%)
Positive Markers

| | | | | |
|---|---|---|---|---|
| CD90+ | 99.90 | 76.33 | 68.29 | 55.02 |
| CD73+ | 98.41 | 75.97 | 68.45 | 56.79 |
| CD105+ | 98.13 | 80.38 | 71.75 | 54.72 |

Negative marker

| | | | | |
|---|---|---|---|---|
| CD34 | 0.19 | 0.33 | 0.16 | 0.09 |
| CD45 | 0.82 | 0.61 | 0.10 | 0.46 |
| HLADR | 0.72 | 0.14 | 0.06 | 0.08 |

Potency (Secretomes estimation)

| | | | | |
|---|---|---|---|---|
| IL10 ng/ml | 957 | 1160 | 940 | 820 |
| VEGF pg/ml | 1714 | 1790 | 1690 | 1580 |

Gene expression (Fold expression) *

| | | | | |
|---|---|---|---|---|
| COL12 A1 | 18.10 | — | — | 10.56 |
| IGFBP5 | 17.80 | — | — | 11.13 |
| THBS2 | 14.90 | — | — | 11.08 |
| GREM1 | 21.00 | — | — | 13.98 |

* Data represents as fold gene expression when compared with GAPDH gene.

The cells when mixed with Hyaluronidase (2000 IU/ml), it shows that the cell recovery is more than 98% at 0 hours of time and 81% at 3 hours of time. The cell goes down at 70% at 6 hours and 64% at 12 hours (Table 7).

Further, cells when treated with hyaluronidase enzyme (2000 IU/ml) exhibit a similar initial viability (over 99% at 0 hours). However, this viability shows a more pronounced decline over time, dropping to 84% at 3 hours, 67% at 6 hours, and 53% at 12 hours.

Annexin V data showed that the cells gradually entered the early apoptotic phase upon mixing with hyaluronidase.

The pH of the solution remained around 6.6 to 6 without much change over time.

The cell marker expression for CD90, CD73 and CD105 also changed with time when cells were mixed with hyaluronidase. There was not much change in the expression of the negative markers CD34, CD45 and HLA-DR.

In the secretomes analysis, secretomes of IL-10 and VEGF increased at 3 hours and showed decrease 6 hour onwards. In the gene expression studies, all the gene expression of the cells decreased at 12 hours when the cells were mixed with hyaluronidase.

Overall, the present disclosure provides important insights into the optimal transport/storage conditions for HUC-MSCs and suggest that RL with 1% HSA, 1 ng/ml serelaxin, 1% dextran 40 and heparin 1000 U/ml is the most suitable carrier in a formulation for the treatment of different autoimmune diseases or fibrotic diseases.

### Advantages of the Present Disclosure

The present disclosure provides a formulation comprising a population of MSCs with therapeutic potential and a carrier specifically comprising Ringer's lactate solution, serelaxin, HSA, heparin, and dextran with the following advantages.
1. The MSCs viability was maintained at 97.54% for up to 24 h.
2. The cell recovery was 97.00% for up to 24 h.
3. Cell characterization for positive cell surface markers showed 95.61% for CD90; 94.72% for CD73 and 93.86% for CD105 for up to 24 h.
4. Cell characterization for negative cell surface markers showed 0.632% for CD34; 0.462% for CD45 and 0.645 for HLA-DR for up to 24 h.
5. The estimated amount of secretomes comprising IL-10 and VEGF was 1002 ng/ml and 1701 μg/ml, respectively for up to 24 h.
6. In gene expression analysis, 16.40-fold expression for COL12A1, 15.50-fold expression for IGFBP5, 15.20-fold expression for THBS2 and 18.25-fold expression for GREM1 was observed for up to 24 h.
7. The MSCs by itself when suspended in an excipient comprising DMEM and HSA in 1:1 ratio showed stability for 48 h after freeze/thaw cycle at 15 to 28° C.
8. The MSCs when as part of the formulation was found to be stable for up to 24 h at 15 to 28° C.

I/We claim:

1. A formulation comprising:
a) a population of mesenchymal stem cells (MSCs); and
b) a carrier selected from serelaxin, Ringer's lactate solution, human serum albumin (HSA), heparin, dextran, or combinations thereof;
wherein said population of MSCs is a homogeneous population having size in the range of 15-30 μm; wherein at least 50% of the MSCs express at least one marker selected from the group consisting of CD 90, CD73, and CD 105.

2. The formulation as claimed in claim 1, wherein the carrier further comprises hyaluronidase.

3. The formulation as claimed in claim 1, wherein the MSCs exhibit increased expression of COL12A1 gene, IGFBP5 gene, THBS2 gene and GREM 1 gene, as compared to the expression of GAPDH gene, wherein the increased expression of COL12A1 is in the range of 14 to 19 folds as compared to the expression of GAPDH gene; wherein the increased expression of IGFBP5 gene is in the range of 14 to 18 folds as compared to the expression of GAPDH gene; wherein the increased expression of THBS2 gene is in the range of 15 to 17 folds as compared to the expression of GAPDH gene; wherein the increased expression of GREM 1 gene is in the range of 18 to 21 folds as compared to the expression of GAPDH gene.

4. The formulation as claimed in claim 1, wherein said formulation comprises secretome of said MSCs comprising VEGF in an amount in the range of 2050 to 2390 μg per million MSCs, and IL-10 in an amount in the range of 1430 to 1690 μg per million MSCs.

5. The formulation as claimed in claim 1, wherein the carrier is in a weight percentage range of 0.5 to 2.5% of the formulation.

6. The formulation as claimed in claim 1, wherein the serelaxin is a 0.5 to 5 ng/ml concentration solution; the Ringer's lactate solution comprises 25 to 30 mM of sodium lactate, 100 to 105 mM of sodium chloride, and 3.5 to 4.5 mM of potassium chloride and 1.5 to 2 mM of calcium chloride; HSA is a 1 to 20% concentration solution; dextran is a 0.5% to 19% concentration solution; heparin is in a concentration range of 500 to 2000 U/ml; and hyaluronidase is in a concentration range of 500 to 5000 U/ml.

7. The formulation as claimed in claim 1, wherein the MSCs are present in the range of 0.5×10⁶ cells/ml to 2×10⁶ cells/ml of the formulation, preferably 1.8×10⁶ cells/ml of the formulation.

8. The formulation as claimed in claim 1, wherein the homogeneous population of MSCs is obtained by artificial intelligence driven microfluidic sorting of culture expanded MSCs, preferably using an artificial intelligence-based tool.

9. The formulation as claimed in claim 1, wherein the MSCs are derived from umbilical cord tissue, cord blood, adipose tissue, bone marrow, or dental pulp.

10. The formulation as claimed in claim 1, wherein the homogeneous population of MSCs are present in a suspension comprising an excipient selected from DMEM, human serum albumin (HSA) or combinations thereof; preferably the excipient is DMEM and 20% HSA solution in a weight ratio in a range of 1:2 to 2:1, more preferably the excipient is DMEM and 20% HSA solution in a weight ratio of 1:1.

11. The formulation as claimed in claim 1, wherein the formulation comprises additives selected from DMEM medium, saline solution, phosphate buffered saine (PBS) buffer, Hank's balanced salt solution (HBSS), human plasma, plasma lysate, or mixtures thereof.

12. The formulation as claimed in claim 1, wherein percentage cell recovery of said MSCs in the formulation estimated after 0 to 24 hours is in the range of 99 to 80% and a percentage cell viability of said MSCs in the formulation estimated after 0 to 24 hours is in the range of 99.8% to 80%.

13. The formulation as claimed in claim 1, wherein the formulation has a pH in the range of 6.4 to 6.6; and wherein the formulation has an osmolarity in the range of 275 to 310 mOsm/L.

14. The formulation as claimed in claim 1, wherein the population of MSCs comprises less than 6% of early apoptotic cells; and less than 3% of late apoptotic cells.

15. A method for preparing the formulation as claimed in claim 1, wherein the method comprises:
mixing the plurality of MSCs with the carrier to obtain the formulation.

16. The method as claimed in claim 15, wherein the method further comprises the addition of one or more additives.

17. A method of inducing tissue regeneration, comprising:
administering to a tissue in need thereof the formulation as claimed in claim 1; wherein the tissue is selected from epithelial tissue; connective tissue like bone tissue, cartilage tissue or elastic tissue; muscle tissue; or nervous tissue.

18. A method of treating autoimmune or fibrotic disease in a subject, comprising:
administering the formulation as claimed in claim 1 to the subject.

19. The method as claimed in claim 18, wherein administering the formulation is via the intravenous, intramuscular, intraosseous, subcutaneous, intraplantar, or intraarticular route.

20. The method as claimed in claim 18, wherein the auto immune disease or fibrotic disease is selected from the group consisting of Acromegaly, Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barre Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, Juvenile polymyositis/Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndrome (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Rheumatoid Arthritis (RA), Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis (TM), Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, ureteric stricture, eye fibrosis, heart fibrosis, hepatic fibrosis, intestinal fibrosis, lung fibrosis, Pancreas fibrosis, renal fibrosis, and skin fibrosis.


SEQUENCE LISTING (8 sequences)

SEQ ID NO: 1 - DNA, 22 bp, synthetic construct
  source 1..22: mol_type = other DNA; note = COL12 A1 Forward Primer
cagtgcctgtagtcagcctgaa

SEQ ID NO: 2 - DNA, 22 bp, synthetic construct
  source 1..22: mol_type = other DNA; note = COL12 A1 Reverse Primer
ggtcttgttggctctgtgtcct

SEQ ID NO: 3 - DNA, 22 bp, synthetic construct
  source 1..22: mol_type = other DNA; note = IGFBP5 Forward Primer
cgtgctgtgtacctgcccaatt

SEQ ID NO: 4 - DNA, 22 bp, synthetic construct
  source 1..22: mol_type = other DNA; note = IGFBP5 Reverse Primer
acttgtccacgcaccagcagat

SEQ ID NO: 5 - DNA, 22 bp, synthetic construct
  source 1..22: mol_type = other DNA; note = THSB2 Forward Primer
cagtctgagcaagtgtgacacc

SEQ ID NO: 6 - DNA, 22 bp, synthetic construct
  source 1..22: mol_type = other DNA; note = THSB2 Reverse Primer
ttgcagagacggatgcgtgtga

SEQ ID NO: 7 - DNA, 23 bp, synthetic construct
  source 1..23: mol_type = other DNA; note = GREM1 Forward Primer
tcatcaaccgcttctgttacggc

SEQ ID NO: 8 - DNA, 22 bp, synthetic construct
  source 1..22: mol_type = other DNA; note = GREM1 Reverse Primer
cagaaggagcaggactgaaagg